(19) United States
(12) Patent Application Publication
(10) Pub. No.: US 2026/0002106 A1
(43) Pub. Date: Jan. 1, 2026

(54) Microfluidic Well Plates and Related Methods

(71) Applicant: The Regents of the University of California, Oakland, CA (US)

(72) Inventors: John Selberg, Santa Cruz, CA (US); Sergio Cordero, Santa Cruz, CA (US); Spencer Seiler, Santa Cruz, CA (US); Mircea Teodorescu, Santa Cruz, CA (US); Marco Rolandi, Santa Cruz, CA (US); Gary Mantalas, Santa Cruz, CA (US)

(21) Appl. No.: 18/707,043

(22) PCT Filed: Nov. 2, 2022
(86) PCT No.: PCT/US2022/048733

§ 371 Date: May 2, 2024

Related U.S. Application Data
(60) Provisional application No. 63/275,136, filed on Nov. 3, 2021.

(51) Int. Cl. C12M 3/06; C12M 1/00; C12M 1/32; C12M 3/00

(52) U.S. Cl. CPC C12M 23/16; C12M 21/08; C12M 23/12; C12M 23/22; C12M 23/38; C12M 29/00; C12M 47/02

(57) ABSTRACT

A cell culture well, cell culture plates and microfluidic systems comprising cell culture wells, and methods of using them for cell culture are provided. Inlets are positioned at the top of the cell culture well to provide enough spacing between the inlet and cell culture to slow fluidic flow over cells and reduce damage to cells. The inlet channel slopes down from the top of the plate to the culture well. The angle and profile of the inlet and outlet geometries are designed to manipulate or eliminate vortices when fluid is flowing. A ramp that directs culture media into the well may also be included. An advantage of the design is that the entire fluidic component can be injection molded or three-dimensionally (3D) printed in a single step.

### CROSS-REFERENCE TO RELATED APPLICATIONS

This application claims the benefit of U.S. Provisional Patent Application No. 63/275,136, filed Nov. 3, 2021, which application is incorporated herein by reference in its entirety.

### BACKGROUND OF THE INVENTION

Systems that perform automated cell culture can enhance pharmacology, pathology, and basic research by improving the quality and consistency of cultures while reducing reagent consumption and required labor. There remains a need for better devices and methods for three-dimensional (3D) culturing of cells (organoids) or patient tissue samples, particularly for basic research and medical applications.

### SUMMARY OF THE INVENTION

A cell culture well, cell culture plates and microfluidic systems comprising cell culture wells, and methods of using them for cell culture are provided. Inlets are positioned at the top of the cell culture well to provide enough spacing between the inlet and cell culture to slow fluidic flow over cells and reduce damage to cells. The inlet channel slopes down from the top of the plate to the culture well. The angle and profile of the inlet and outlet geometries are designed to manipulate or eliminate vortices when fluid is flowing. A ramp that directs culture media into the well may also be included. An advantage of the design is that the entire fluidic component can be injection molded or three-dimensionally (3D) printed in a single step.

In one aspect, a cell culture well is provided, the cell culture well comprising: a chamber comprising a bottom surface and a top opening; an inlet channel in fluid communication with the chamber, wherein the inlet channel connects with the chamber at the top opening at a first side of the chamber, wherein a first segment of the inlet channel is substantially perpendicular to the first side at the bottom surface, and wherein a second segment of the inlet channel is substantially parallel to the first side of the chamber from the top opening to the bottom surface; and an outlet channel in fluid communication with the chamber, wherein the outlet channel connects with the chamber at the top opening, wherein a first segment of the outlet channel is substantially parallel to a second side of the chamber from the top opening to the bottom surface, and wherein a second segment of the outlet channel is substantially perpendicular to the second side at the bottom surface.

In certain embodiments, the inlet channel is configured to be substantially perpendicular to the first side for a first distance and substantially parallel to the first side at the end of the first distance and wherein the outlet channel is configured to be substantially perpendicular to the second side for a second distance and substantially parallel to the first side at the end of the first distance.

In certain embodiments, the first side comprises a first ramp portion on top of the first side, and the second side comprises a second ramp portion on top of the second side, wherein the first ramp portion forms an obtuse angle relative to the first side and the second ramp portion forms an obtuse angle relative to the second side.

In certain embodiments, the cell culture well further comprises a cap, wherein the top opening is covered with the cap. In some embodiments, the cap is removable.

In certain embodiments, the bottom surface is transparent (e.g., to allow optical monitoring of cultures from the bottom).

In another aspect, a cell culture plate comprising at least two of the cell culture wells, described herein, is provided.

In certain embodiments, the inlet channel of a first cell culture well is in fluid communication with the inlet channel of a second cell culture well.

In certain embodiments, the outlet channel of a first cell culture well is in fluid communication with the outlet channel of a second cell culture well.

In certain embodiments, the cell culture plate comprises at least 2, at least 4, at least 6, at least 12, at least 24, at least 48, at least 96, at least 384, or at least 1536 cell culture wells.

In another aspect, a microfluidic device comprising a cell culture well or a cell culture plate, described herein, is provided.

In certain embodiments, the microfluidic device further comprises a container for storage of culture media. The storage container can be connected to an inlet channel to flow the culture media into a cell culture well in the device.

In certain embodiments, the microfluidic device further comprises a container for collection of waste.

In certain embodiments, the microfluidic device further comprises a pump to control the flow rate of media through the device. Any suitable type of pump may be used, including without limitation, a syringe pump, peristaltic pump, or pressure driven pump.

Additionally, microfluidic devices described herein can be adapted for growing multiple cultures in parallel. For example, the microfluidic device may comprise multiple cell culture wells with chambers for growing cultures. In some embodiments, the microfluidic device comprises a plurality of cell culture wells, wherein the chamber in each of the cell culture wells is configured to contain a volume of culture media. In some embodiments, the chamber in each cell culture well is in fluid communication with an inlet channel and an outlet channel configured to allow the flow of media between the chambers while preventing movement of cells between the chambers of the different cell culture wells, wherein the flow of media is facilitated by a pump. In certain embodiments, the microfluidic device further comprises a flow rate sensor.

In certain embodiments, the microfluidic device further comprises a multiport fluid distribution valve to direct the flow of media through the device. The multiport fluid distribution valve can be used to control fluid routing and direct media flow to a particular cell culture well or group of cell culture wells in the microfluidic device.

In certain embodiments, the microfluidic device further comprises a temperature sensor and temperature control unit to maintain the temperature of the cultures in a suitable range.

A cell culture well, as described herein, may be used to culture, for example, a cell, a population of cells, a tissue, an organoid, or a non-human organism. In a cell culture plate or microfluidic device comprising multiple cell culture wells, each of the cell culture wells may comprise the same type of cell, population of cells, tissue, organoid, or non-human organism. Alternatively, each of the cell culture wells may comprise different types of cells, populations of cells, tissues, organoids, or non-human organisms.

A cell, population of cells, tissue, organoid, or non-human organism can be introduced into the chamber through the top opening. Culture media is introduced into the inlet channel and flows through the inlet channel into the chamber where the cell, population of cells, tissue, organoid, or non-human organism is cultured in the culture media under conditions suitable for growth of the cell, population of cells, tissue, organoid, or non-human organism. In certain embodiments, the culture media is exchanged by flowing the conditioned culture media through the outlet channel, for example, into a waste container and flowing new media through the inlet channel into the chamber. In addition, media supplements may be added by flowing them through the inlet channel into the chamber. Culture media and media supplements may be stored in one or more reservoirs connected to the inlet channel. A multiport fluid distribution valve can be used to control which media or supplement is added to a chamber. In some embodiments, factors secreted from a cell, population of cells, tissue, or organoid in one chamber flow to a cell, population of cells, tissue, or organoid in one or more other chambers. After culturing is completed, the cell, tissue, organoid, or non-human organism may be removed from the chamber.

In some embodiments, one or more of the cells, population of cells, tissues, organoids, or non-human organisms may be analyzed before, during, or after culturing with an analytical instrument. In some embodiments, the analytical instrument is a microscope, imaging device, or fluorimeter. In certain embodiments, the bottom of the chamber is transparent to facilitate microscopic visualization of cells, imaging, or luminescent, fluorescent, or colorimetric assays of cultures contained in the chamber. Confluency, morphology, and other parameters may be monitored during culturing.

In some embodiments, the cells, population of cells, tissues, organoids, or non-human organisms are analyzed to determine their response to exposure to a test agent. Candidate test agents may include, without limitation, pharmaceutical agents such as small molecules, drugs, chemotherapeutic agents, biologic agents, and immunotherapeutic agents, antibodies, peptides, proteins, secreted factors such as growth factors, cytokines, chemokines, and hormones, and genetic agents such as antisense nucleic acids, miRNA, siRNA, shRNA, mRNA, cDNA, CRISPR systems, vectors encoding expressible sequences, toxins, pathogenic agents such as viruses, bacteria, fungi, protists, and the like. In some embodiments, a cell, tissue, organoid, or non-human organism, grown in a cell culture well as described herein, may be used as a disease model to determine the effect of a treatment of a condition with a test agent. Methods are also provided for screening for agents that modulate tissue or organ function using the cell, tissue, organoid, or non-human organism grown in the cell culture well. Cell culture conditions may also be screened to determine how a change in growth conditions (e.g., change of temperature, media, or supplements) effects growth of a cell, tissue, organoid, or non-human organism grown in the cell culture well. In certain embodiments, expression profiling or a biochemical assay may be performed on a cell, population of cells, tissue, organoid, or non-human organism.

In another aspect, a method of making a cell culture well, described herein, is provided, the method comprising: providing a first layer comprising a set of channels parallel to the plane of the first layer; and adding on top of the first layer, a second layer, the second layer comprising a hole, an inlet chamber in fluid communication with an end of the hole configured to be furthest from the first layer and an outlet channel in fluid communication with the end of the hole configured to be furthest from the first layer.

In another aspect, an injection molding for fabricating a cell culture well, described herein, is provided, the injection molding comprising a substrate surrounding a cavity having a configuration as illustrated in FIG. 5, wherein material injected into the cavity hardens to assume the configuration of the cavity to form a molded cell culture well.

### BRIEF DESCRIPTION OF THE DRAWINGS

FIG. 1. Schematic of microfluidic well plate showing the inlet channel, outlet channel, ramps, and culture well.

FIG. 2. Overview of the full system. Components-Conditioned media and storage, fluid pump, multiport fluid distribution valve, microfluidic autoculture chip, flow rate sensor, and waste media storage.

FIGS. 3A-3F. Organoid Plate Geometry. FIG. 3A Open-well organoid plate geometry. FIG. 3B: Two components of the organoid plate, a machined piece (top) and a base piece (bottom). FIG. 3C: Vertical fluid line connecting to surface of plate and the ramp directing media into the well. FIG. 3D: Photo of an early version of this geometry. FIG. 3E: prior art bioreactor with closed lines entering the well. FIG. 3F) Organoid chip with a removable cap (dark grey).

FIGS. 4A-4B. Open-Well Fluidics with Samples. FIG. 4A: Organoids cultured on a chip. FIG. 4B: Mouse brain tissue cultured on a chip.

FIG. 5. Injection molding for producing microfluidic well plates.

### DETAILED DESCRIPTION

A cell culture well, cell culture plates and microfluidic systems comprising cell culture wells, and methods of using them for cell culture are provided. Inlets are positioned at the top of the cell culture well to provide enough spacing between the inlet and cell culture to slow fluidic flow over cells and reduce damage to cells. The inlet channel slopes down from the top of the plate to the culture well. The angle and profile of the inlet and outlet geometries are designed to manipulate or eliminate vortices when fluid is flowing. A ramp that directs culture media into the well may also be included. An advantage of the design is that the entire fluidic component can be injection molded or three-dimensionally (3D) printed in a single step.

Before the present cell culture well, cell culture plates, and microfluidic systems and methods of using them for cell culture are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the organoid” includes reference to one or more organoids and equivalents thereof, e.g., three-dimensional tissue cultures and explants comprising organ-specific cell types known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The term “cell culture” or “culture” refers to the maintenance of cells in an artificial, in vitro environment. It is to be understood, however, that the term “cell culture” is a generic term and may be used to encompass the cultivation not only of individual cells, but also of tissues, organs, organoids, and non-human organisms.

The term “explant” is used herein to mean a piece of tissue and the cells thereof originating from mammalian tissue that is cultured in vitro, for example in a cell culture well, as described herein. The mammalian tissue from which the explant is derived may be obtained from an individual, i.e., a primary explant, or it may be obtained in vitro, e.g., by differentiation of induced pluripotent stem cells.

The term “organoid” as used herein refers to a 3-dimensional growth of mammalian cells in culture that retains characteristics of a tissue in vivo, e.g., prolonged tissue expansion with proliferation, multilineage differentiation, recapitulation of cellular and tissue ultrastructure, etc. A primary organoid is an organoid that is cultured from an explant, i.e., a cultured explant. A secondary organoid is an organoid that is cultured from a subset of cells of a primary organoid, i.e., the primary organoid is fragmented, e.g., by mechanical or chemical means, and the fragments are replated and cultured. A tertiary organoid is an organoid that is cultured from a secondary organoid, etc.

The phrase “mammalian cell” refers to any cell originating from mammalian tissue. The cell can be a primary cell obtained directly from a mammalian subject. The cell may also be a cell derived from the culture and expansion of a cell obtained from a subject. For example, the cell may be a stem cell, progenitor cell, or adult cell. Immortalized cells are also included within this definition. In some embodiments, the cell has been genetically engineered to express a recombinant protein and/or nucleic acid. The term “mammalian” includes, without limitation, human, equine, bovine, porcine, canine, feline, rodent (e.g., mice, rats, hamster), and primate.

“Biocompatible,” as used herein, refers to a property of a material that allows for prolonged contact with a tissue in a subject without causing toxicity or significant damage.

### Cell Culture Wells

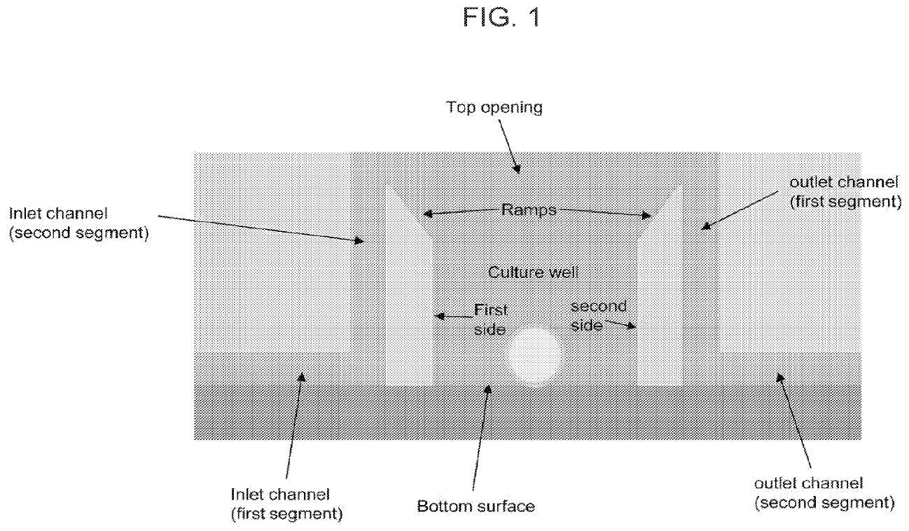

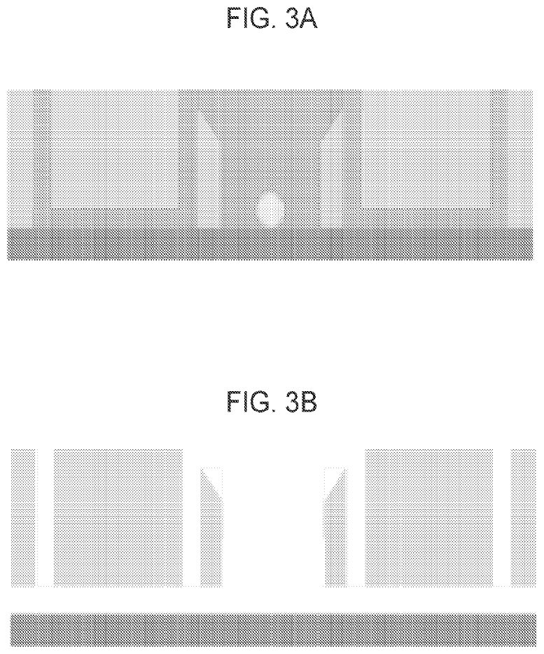

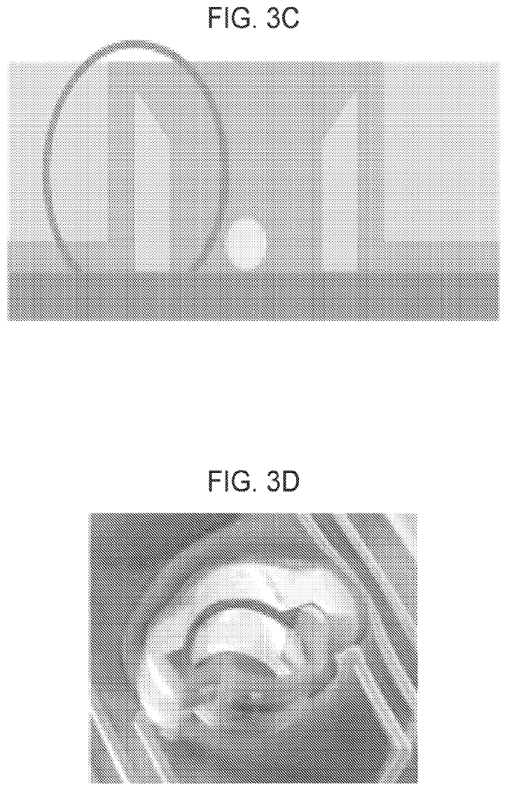

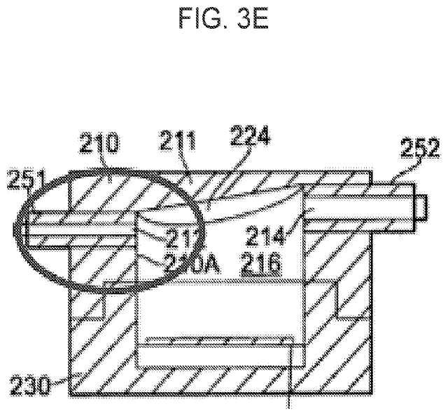

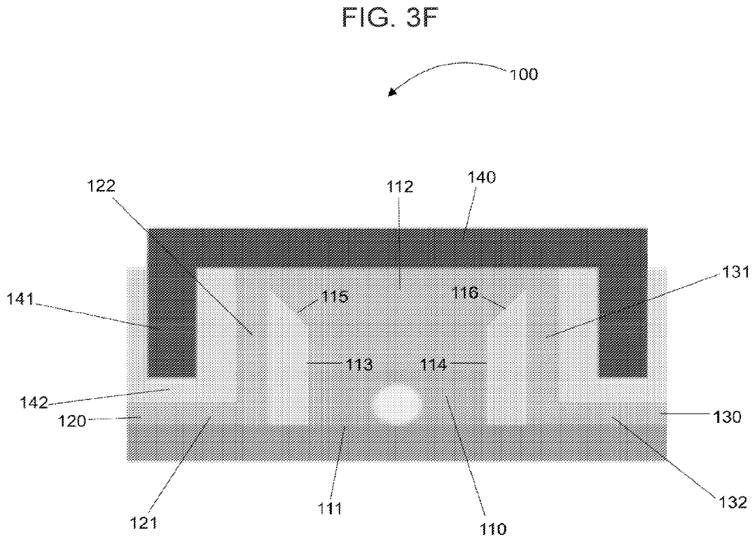

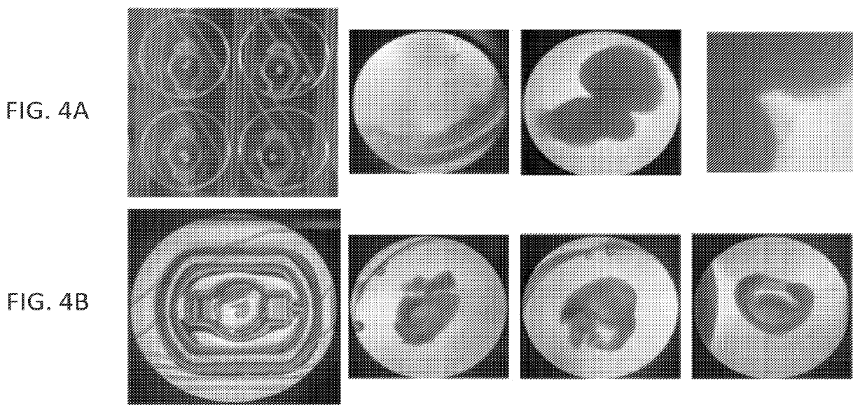

Exemplary cell culture wells are shown in FIG. 1 and FIG. 3F and described in the Examples. A cell culture well 100 comprises: a chamber 110 comprising a bottom surface 111 and a top opening 112; an inlet channel 120 in fluid communication with the chamber 110, wherein the inlet channel 120 connects with the chamber 110 at the top opening 112 at a first side 113 of the chamber 110, wherein a first segment 121 of the inlet channel 120 is substantially perpendicular to the first side 113 at the bottom surface 111, and wherein a second segment 122 of the inlet channel 120 is substantially parallel to the first side 113 of the chamber 110 from the top opening 112 to the bottom surface 111; and an outlet channel 130 in fluid communication with the chamber 110, wherein the outlet channel 130 connects with the chamber 110 at the top opening 112, wherein a first segment 131 of the outlet channel 130 is substantially parallel to a second side 114 of the chamber 110 from the top opening 112 to the bottom surface 111, and wherein a second segment 132 of the outlet channel 130 is substantially perpendicular to the second side 114 at the bottom surface 111. In certain embodiments, the inlet channel 120 is configured to be substantially perpendicular to the first side 113 for a first distance and substantially parallel to the first side 113 at the end of the first distance and where the outlet channel 130 is configured to be substantially perpendicular to the second side 114 for a second distance and substantially parallel to the first side 113 at the end of the first distance.

Inlets are positioned at the top of the cell culture well to provide enough spacing between the inlet and cell culture to slow fluidic flow over cells and reduce damage to cells. The inlet channel slopes down from the top of the plate to the culture well. The angle and profile of the inlet and outlet geometries are designed to manipulate or eliminate vortices when fluid is flowing.

In certain embodiments, the first side 113 comprises a first ramp portion 115 on top of the first side 113, and the second side 114 comprises a second ramp portion 116 on top of the second side 114, wherein the first ramp portion forms an obtuse angle relative to the first side and the second ramp portion forms an obtuse angle relative to the second side.

In certain embodiments, the cell culture well further comprises a removable cap, wherein the top opening can be covered with the cap. FIG. 3F shows an exemplary cap 140 that is sized and shaped to engage the cell culture well 100. In some embodiments, the cap 140 has projections 141 that fit into a complementary receiving location 142 of the cell culture well 100.

In certain embodiments, the bottom surface 111 is transparent to allow optical monitoring of cultures from the bottom. The bottom surface may be constructed, for example, from transparent materials such as, but not limited to, transparent polystyrene, cyclic olefin copolymer, cyclic olefin polymer, or quartz. In some embodiments, the transparent bottom is used for microscopic visualization of cells, imaging, or luminescent, fluorescent, or colorimetric assays of cultures contained in the chamber 110. Confluency, morphology, and other parameters may be monitored during culturing.

### Cell Culture Plates

In another aspect, a cell culture plate comprising one or more cell culture wells, as described herein, is provided. The cell culture plate may have various shapes such as, but not limited to, rectangular, square, circular, semicircular, oval, or triangular. In some embodiments, the cell culture plate has dimensions and a distribution of cell culture wells substantially the same as commercially available multi-well plates for commercially available plate readers. For example, a cell culture plate comprising multiple cell culture wells, as described herein, may have a substantially rectangular shape with a spacing of cell culture wells appropriate for commercially available plate readers and dispensers. In other embodiments, the cell culture plate has a shape that is not rectangular.

In certain embodiments, the cell culture plate comprises at least 1, at least 2, at least 4, at least 6, at least 12, at least 24, at least 48, at least 96, at least 384, or at least 1536 cell culture wells. In some embodiments, the cell culture plate comprises between 1 and 1536 cell culture wells, including any number of cell culture wells within this range, such as 1, 2, 3, 4, 5, 6, 7, 8, 9, 10, 11,12, 13, 14, 15, 16, 17, 18, 19, 20, 21, 22, 23, 24, 48, 96, 144, 192, 240, 288, 336, 384, 432, 480, 528, 576, 624, 672, 720, 768, 816, 864, 912, 960, 1008, 1056, 1104, 1152, 1200, 1248, 1296, 1344, 1392, 1440, 1488, or 1536 cell culture wells per cell culture plate.

In certain embodiments, at least two cell culture wells in a cell culture plate are fluidically connected. In some embodiments, the inlet channel of a first cell culture well is in fluid communication with the inlet channel of a second cell culture well or a plurality of cell culture wells. In certain embodiments, the outlet channel of a first cell culture well is in fluid communication with the outlet channel of a second cell culture well or a plurality of cell culture wells.

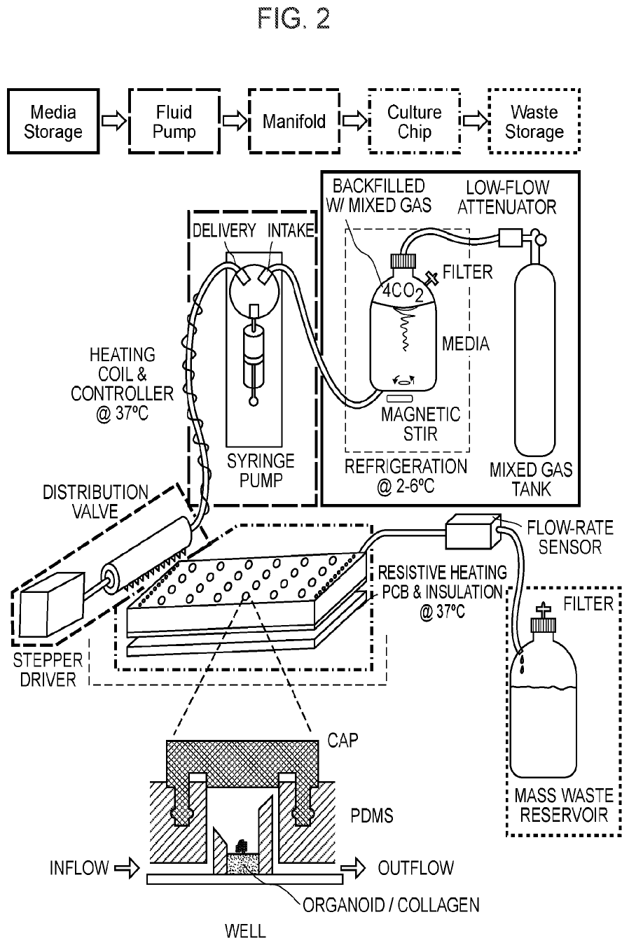

An advantage of the design is that a two-step process can be used for manufacturing microfluidic plates comprising the cell culture wells (see Examples). First, a machined layer defining the microchannels and well geometry is produced. Next, the machined layer is bonded to a substrate to seal the microchannels (see, e.g., FIG. 3B). The fluidic channel that connects the fluidics at the bottom of the plate to the inlet at the top of the well is a vertical channel. The inlet to the cell culture well includes a sloping channel from the top of the plate to the well itself (FIGS. 2C, 2D).

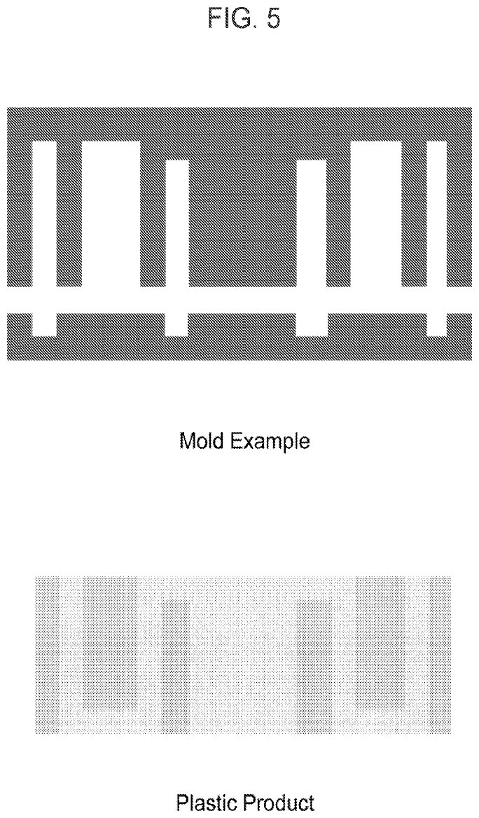

An exemplary injection molding for fabricating a cell culture well is shown in FIG. 5. The injection molding comprises a substrate surrounding a cavity having a configuration that provides a negative representation of the features of the cell culture well. Material, injected into the cavity, hardens to assume the configuration of the cavity to form a molded cell culture well.

Injection molding can be performed with molten metals, glasses, elastomers, or thermoplastic polymers. In some embodiments, the cell culture plate is manufactured from polymeric materials such as, but not limited to, polymeric organosilicon compounds (e.g., polydimethylsiloxane), hydrophilic polyethylenes, polystyrenes, polypropylenes, acrylates, methacrylates, polycarbonates, polysulfones, polyesterketones, poly-or cyclic olefins, polychlorotrifluoroethylene, polyethylene therephthalate, and inorganic polymer materials. In some embodiments, the cell culture plate is manufactured from thermoplastic elastomers such as, but not limited to thermoplastic styrenic block copolymers, polyolefinelastomers, vulcanizates, polyurethanes, copolyester, and polyamides.

### Microfluidic Devices

In another aspect, a microfluidic device comprising a cell culture plate or cell culture well, as described herein, is provided. In some embodiments, the microfluidic device comprises a series of channels, valves, and microelectromechanical pumps that transport media from an entry point of the device through an inlet channel to a chamber of a cell culture well. Media can be removed from a chamber through an outlet channel after completion of culturing or to allow media exchanges during culturing. The cell culture wells, channels, valves, and pumps may be manufactured on a single substrate, which may be elastomeric or metallic in nature. Soft lithography techniques are typically used for an elastomeric substrate, whereas photolithography or electron beam lithography can be used for the patterning of metallic or semiconductor substrates.

In certain embodiments, the microfluidic device comprises a pump to control the flow rate of media through the device. Any suitable type of pump may be used, including without limitation, a syringe pump, peristaltic pump or pressure driven pump. In some embodiments, the microfluidic device further comprises a flow rate sensor.

The microfluidic device can be adapted for growing multiple cultures in parallel. For example, the microfluidic device may comprise multiple cell culture wells with chambers for growing cultures, as described herein. In some embodiments, the microfluidic device comprises a plurality of cell culture wells, wherein the chamber in each of the cell culture wells is configured to contain a volume of culture media. In some embodiments, the chamber in each cell culture well is in fluid communication with an inlet channel and an outlet channel configured to allow the flow of media between the chambers while preventing movement of cells between the chambers of the different cell culture wells, wherein the flow of media is facilitated by the pump.

In certain embodiments, the microfluidic device further comprises a multiport fluid distribution valve to direct the flow of media through the device. The multiport fluid distribution valve can be used to control fluid routing and direct media flow to a particular cell culture well or group of cell culture wells in the microfluidic device.

A cell culture well, as described herein, may be used to culture, for example, a cell, a population of cells, a tissue, an organoid, or a non-human organism. In some embodiments, each of the cell culture wells in the microfluidic device comprise the same type of cell, population of cells, tissue, organoid, or non-human organism. In other embodiments, each of the cell culture wells comprise different types of cells, populations of cells, tissues, organoids, or non-human organisms.

A cell, population of cells, tissue, organoid, or non-human organism can be introduced into the chamber of a cell culture well through the top opening. Culture media flows through the inlet channel into the chamber where the cell, population of cells, tissue, organoid, or non-human organism is cultured under conditions suitable for growth of the cell, population of cells, tissue, organoid, or non-human organism. In certain embodiments, the microfluidic device further comprises a temperature sensor and temperature control unit to maintain the temperature of the cultures in a suitable range. After culturing is completed, the cell, tissue, organoid, or non-human organism can be removed from the chamber.

Culture media can be exchanged during culturing by flowing conditioned culture media out of the chamber of a cell culture well through the outlet channel and out of the device, e.g., into a waste container, and flowing new media through the inlet channel into the chamber. In addition, media supplements may be added by flowing them through the inlet channel into the chamber. Culture media and media supplements may be stored in one or more reservoirs connected to the inlet channel. A multiport fluid distribution valve can be used to control which media or supplement is added to a chamber.

In some embodiments, the microfluidic device has a single channel connecting two cell culture wells. In certain embodiments, the inlet channel of a first cell culture well is in fluid communication with the inlet channel of a second cell culture well. In certain embodiments, the outlet channel of a first cell culture well is in fluid communication with the outlet channel of a second cell culture well. In some embodiments, factors secreted from a cell, population of cells, tissue, or organoid in one chamber flow to a cell, population of cells, tissue, or organoid in one or more other chambers.

### Cultures

In some embodiments, cells or tissue are obtained from a subject for the purpose of growing cultures of cells, populations of cells, tissue, or organoids in a cell culture well, as described herein. The cells may be derived from any tissue, including connective tissue, muscle tissue, nervous tissue, or epithelial tissue. Cells or tissue may be obtained by any convenient method including, without limitation, by biopsy, e.g., during endoscopy, during surgery, by needle, etc., and are preferably obtained as aseptically as possible. In some embodiments, the cells or tissue are from a mammalian species such as, but not limited to a human, equine, bovine, porcine, canine, feline, rodent (e.g., mice, rats, hamster), or primate subject. The subject may be of any age, e.g., a fetus, neonate, juvenile, or adult.

Cells used in cultures can be primary cells obtained directly from a subject. Alternatively, the cells may be derived from the culture and expansion of a cell obtained from a subject or a cell obtained from a cell line. In some embodiments, the cell is an adult cell. In other embodiments, the cell is a progenitor cell or stem cell, or a differentiated cell derived from a progenitor cell or stem cell. Immortalized cells may also be used in cultures. In some embodiments, the cell has been genetically engineered to express a recombinant protein and/or nucleic acid.

Cells or tissue used in cultures may be obtained from any part of the body of a subject, including, without limitation, from the cardiovascular system, including the heart, blood, blood vessels, and lungs; digestive system, including the salivary glands, esophagus, stomach, liver, gallbladder, pancreas, intestines, colon, rectum and anus; endocrine system, including the endocrine glands such as the hypothalamus, pituitary gland, pineal body or pineal gland, thyroid, parathyroids and adrenals (adrenal glands); excretory system, including kidneys, ureters, bladder and urethra involved in fluid balance, electrolyte balance and excretion of urine; lymphatic system, including structures involved in the transfer of lymph between tissues and the blood stream, the lymph and the nodes and vessels that transport it, the immune system, including leukocytes, tonsils, adenoids, thymus and spleen; integumentary system, including skin, hair and nails of mammals, and scales of fish, reptiles, and birds, and feathers of birds; muscular system, including skeletal, smooth and cardiac muscles; nervous system, including the brain, spinal cord, nerves, and glia; reproductive system, including the sex organs, such as ovaries, fallopian tubes, uterus, vulva, vagina, testes, vas deferens, seminal vesicles, prostate and penis; respiratory system, including the organs used for breathing, the pharynx, larynx, trachea, bronchi, lungs and diaphragm; skeletal system, including bones, cartilage, ligaments and tendons.

Cells included in cultures may be of any type such as, but not limited to, exocrine secretory epithelial cells such as a Brunner's gland cell in the duodenum, insulated goblet cell of respiratory and digestive tracts, stomach cells such as foveolar cell (mucus secretion), a chief cell (pepsinogen secretion), parietal cell (hydrochloric acid secretion), and pancreatic acinar cell; a paneth cell of the small intestine, a type II pneumocyte of lung, a club cell of the lung; barrier cells such as a type I pneumocyte (lung), gall bladder epithelial cell, centroacinar cell (pancreas), intercalated duct cell (pancreas), and intestinal brush border cell (with microvilli); hormone-secreting cells such as an enteroendocrine cell, K cell, L cell, I cell, G cell, enterochromaffin cell, enterochromaffin-like cell, N cell, S cell, D cell, Mo cell, thyroid gland cells, thyroid epithelial cell, parafollicular cell, parathyroid gland cells, parathyroid chief cell, oxyphil cell, pancreatic islets (islets of Langerhans), alpha cell (secretes glucagon), beta cell (secretes insulin and amylin), delta cell (secretes somatostatin), epsilon cell (secretes ghrelin), pp cell (gamma cell), cells derived primarily from ectoderm such as exocrine secretory epithelial cells, salivary gland mucous cell, salivary gland serous cell, von Ebner's gland cell in tongue, mammary gland cell, lacrimal gland cell, ceruminous gland cell in ear, eccrine sweat gland dark cell, eccrine sweat gland clear cell, apocrine sweat gland cell, gland of moll cell in eyelid, sebaceous gland cell, and bowman's gland cell in nose; hormone-secreting cells such as anterior/intermediate pituitary cells, corticotropes, gonadotropes, lactotropes, melanotropes, somatotropes, thyrotropes, magnocellular neurosecretory cells, parvocellular neurosecretory cells, and chromaffin cells (adrenal gland); epithelial cells such as a keratinocyte, epidermal basal cell, melanocyte, trichocyte, medullary hair shaft cell, cortical hair shaft cell, cuticular hair shaft cell, Huxley's layer hair root sheath cell, Henle's layer hair root sheath cell, outer root sheath hair cell, surface epithelial cell of cornea, tongue, mouth, nasal cavity, distal anal canal, distal urethra, and distal vagina, basal cell (stem cell) of cornea, tongue, mouth, nasal cavity, distal anal canal, distal urethra, and distal vagina, intercalated duct cell (salivary glands), striated duct cell (salivary glands), lactiferous duct cell (mammary glands), ameloblast, oral cells such as an odontoblast and cementoblast; nervous system cells such as neurons, sensory transducer cells such as auditory inner hair cells of organ of Corti, auditory outer hair cells of organ of Corti, basal cells of olfactory epithelium, cold-sensitive primary sensory neurons, heat-sensitive primary sensory neurons, merkel cells of epidermis, olfactory receptor neurons, pain-sensitive primary sensory neurons, photoreceptor cells of retina in the eye such as photoreceptor rod cells, photoreceptor blue-sensitive cone cells of eye, photoreceptor green-sensitive cone cells of eye, and photoreceptor red-sensitive cone cells of eye; proprioceptive primary sensory neurons, touch-sensitive primary sensory neurons, chemoreceptor glomus cells of carotid body cell, outer hair cells of vestibular system of ear, inner hair cells of vestibular system of ear, taste receptor cells of taste bud, autonomic neuron cells, cholinergic neurons, adrenergic neural cells, peptidergic neural cells, sense organ and peripheral neuron supporting cells, inner pillar cells of organ of Corti, outer pillar cells of organ of Corti, inner phalangeal cells of organ of Corti, outer phalangeal cells of organ of Corti, border cells of organ of Corti, Hensen's cells of organ of Corti, vestibular apparatus supporting cells, taste bud supporting cells, olfactory epithelium supporting cells, olfactory ensheathing cells, schwann cells, satellite glial cells, enteric glial cells, central nervous system neurons and glial cells, interneurons basket cells, cartwheel cells, stellate cells, golgi cells, granule cells, lugaro cells, unipolar brush cells, martinotti cells,, chandelier cells, Cajal-Retzius cells, double-bouquet cells, neurogliaform cells, retina horizontal cells, amacrine cells, starburst amacrine cells, spinal interneurons, renshaw cells, principal cells, spindle neurons, fork neurons, pyramidal cells, place cells, grid cells, speed cells, head direction cells, betz cells, stellate cells, boundary cells, bushy cells, Purkinje cells, medium spiny neurons, astrocytes, oligodendrocytes, ependymal cells, tanycytes, pituicytes, lens cells, anterior lens epithelial cell, crystallin-containing lens fiber cell; metabolism and storage cells such as adipocytes: , white fat cell, brown fat cell, and liver lipocyte; secretory cells such as cells of the adrenal cortex, cells of the zona glomerulosa produce mineralocorticoids, cells of the zona fasciculata produce glucocorticoids, cells of the zona reticularis produce androgens, theca interna cell of ovarian follicle secreting estrogen, corpus luteum cell of ruptured ovarian follicle secreting progesterone, granulosa lutein cells, theca lutein cells, leydig cell of testes secreting testosterone, seminal vesicle cell, prostate gland cell, bulbourethral gland cell, Bartholin's gland cell, gland of littre cell, uterus endometrium cell, juxtaglomerular cell, macula densa cell of kidney, peripolar cell of kidney, and mesangial cell of kidney; urinary system cells such as parietal epithelial cell, podocyte, proximal tubule brush border cell, loop of henle thin segment cell, kidney distal tubule cell, kidney collecting duct cell, principal cell, intercalated cell, and transitional epithelium (lining urinary bladder); reproductive system cells such as duct cell (of seminal vesicle, prostate gland, etc.), efferent ducts cell epididymal principal cell, and epididymal basal cell; circulatory system cells, endothelial cells, extracellular matrix cells, planum semilunatum epithelial cell of vestibular system of ear, organ of Corti interdental epithelial cell, loose connective tissue fibroblasts, corneal fibroblasts (corneal keratocytes) tendon fibroblasts, bone marrow reticular tissue fibroblasts, other nonepithelial fibroblasts, pericyte, hepatic stellate cell (ito cell), nucleus pulposus cell of intervertebral disc, hyaline cartilage chondrocyte, fibrocartilage chondrocyte, elastic cartilage chondrocyte, osteoblast/osteocyte, osteoprogenitor cell, hyalocyte of vitreous body of eye, stellate cell of perilymphatic space of ear, and pancreatic stellate cell; contractile cells such as skeletal muscle cells, red skeletal muscle cell (slow twitch), white skeletal muscle cell (fast twitch), intermediate skeletal muscle cell, nuclear bag cell of muscle spindle, nuclear chain cell of muscle spindle, myosatellite cell (stem cell), cardiac muscle cells, cardiac muscle cell, SA node cell, Purkinje fiber cell, smooth muscle cell (various types) myoepithelial cell of iris myoepithelial cell of exocrine glands; blood and immune system cells such as an erythrocyte (red blood cell) and precursor erythroblasts megakaryocyte (platelet precursor) platelets, a monocyte, connective tissue macrophage (various types), epidermal langerhans cell osteoclast (in bone), dendritic cell (in lymphoid tissues), microglial cell (in central nervous system), neutrophil granulocyte and precursors (myeloblast, promyelocyte, myelocyte, metamyelocyte), an eosinophil granulocyte and precursors basophil granulocyte and precursors, a mast cell, helper T cell, regulatory T cell, cytotoxic T cell, natural killer T cell, B cell, plasma cell, natural killer cell, and hematopoietic stem cells; germ cells such as an oogonium/oocyte, spermatid, spermatocyte, spermatogonium cell, spermatozoon, nurse cell, granulosa cell, sertoli cell, and epithelial reticular cell; and interstitial cells such as interstitial kidney cells.

In some embodiments, the cells are stem cells or stem cell-derived cells. Stem cells of interest include, without limitation, hematopoietic stem cells, embryonic stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, hematopoietic stem cells, induced pluripotent stem cells; and the like; as well as differentiated cells that can be cultured in vitro and used in a therapeutic regimen, where such cells include, but are not limited to, keratinocytes, adipocytes, cardiomyocytes, neurons, osteoblasts, pancreatic islet cells, retinal cells, and the like.

Suitable human embryonic stem(ES) cells include, but are not limited to, any of a variety of available human ES lines, e.g., BG01 (hESBGN-01), BG02 (hESBGN-02), BG03 (hESBGN-03) (BresaGen, Inc.; Athens, Ga.); SA01 (Sahlgrenska 1), SA02 (Sahlgrenska 2) (Cellartis AB; Goeteborg, Sweden); ES01 (HES-1), ES01 (HES-2), ES03 (HES-3), ES04 (HES-4), ES05 (HES-5), ES06 (HES-6) (ES Cell International; Singapore); UC01 (HSF-1), UC06 (HSF-6) (University of California, San Francisco; San Francisco, Calif.); WA01 (H1), WA07 (H7), WA09 (H9), WA09/Oct4D10 (H9-hOct4-pGZ), WA13 (H13), WA14 (H14) (Wisconsin Alumni Research Foundation; WARF; Madison, Wis.). Cell line designations are given as the National Institutes of Health (NIII) code, followed in parentheses by the provider code.

Hematopoietic stem cells (HSCs) are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

Mesenchymal stem cells (MSC), originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC.

An induced pluripotent stem (iPS) cells is a pluripotent stem cell induced from a somatic cell, e.g., a differentiated somatic cell. iPS cells are capable of self-renewal and differentiation into cell fate-committed stem cells, including neural stem cells, as well as various types of mature cells. iPS cells can be generated from somatic cells, including skin fibroblasts, using, e.g., known methods. iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and Klf4. In some embodiments, somatic cells are genetically modified with one or more expression constructs comprising nucleotide sequences encoding Oct-4, Sox2, Nanog, and LIN28. Methods of generating iPS are known in the art, and any such method can be used to generate iPS.

In some cases, the cells are lymphocytes, such as CD4+ and/or CD8+ T lymphocytes, or B lymphocytes. In some embodiments, the therapeutic cells are cytotoxic T lymphocytes. In some embodiments, the lymphocytes are genetically modified lymphocytes, e.g., chimeric antigen receptor (CAR) T lymphocytes. The lymphocytes, e.g., cytotoxic T lymphocytes, may specifically recognize an antigen that is associated with a disease, e.g., cancer or tumor.

In some embodiments, the cells include insulin-secreting cells. The insulin-secreting cells may be any suitable type of insulin-secreting cell. In some cases, the insulin-secreting cells are a type of cell that secretes insulin (e.g., pancreatic β islet cells, or β-like cells). In some cases, the insulin-secreting cells are primary β islet cells (e.g., mature β islet cells isolated from a pancreas). In some cases, the insulin-secreting cells are β cells, or β-like cells that are derived in vitro from immature cell, precursor cells, progenitor cells, or stem cells. The insulin-secreting cells may be derived from (i.e., obtained by differentiating) stem and/or progenitor cells such as hepatocytes (e.g., transdifferentiated hepatocytes), acinar cells, pancreatic duct cells, stem cells, embryonic stem cells(ES), partially differentiated stem cells, non-pluripotent stem cells, pluripotent stem cells, induced pluripotent stem cells (iPS cells), etc. Suitable insulin-secreting cells and methods of generating the same are described in, e.g., US20030082810; US20120141436; and Raikwar et al. (PLOS One. 2015 Jan. 28;10 (1): e0116582), each of which are incorporated herein by reference.

Various culture media, methods of generating tissue explants and organoids, and methods of culturing cells, tissue, and organoids are known in the art. See, e.g., Methods in Enzymology Volume 58 on Cell Culture, edited by N. P. Kaplan, N. P. Colowick, W. B. Jakoby, and I. H. Pastan, Academic Press, 1^{st} edition, 1979; Freshney and Capes-Davis, Freshney's Culture of Animal Cells: A Manual of Basic Technique and Specialized Applications, Wiley-Blackwell, 8^{th} edition, 2021; 3D Cell Culture: Methods and Protocols (Methods in Molecular Biology, 695), edited by J. Haycock, Humana, 2011^{th} edition, 2010; Organoids and Mini-Organs, edited by J. Davies and M. Lawrence, Academic Press, 1^{st} edition, 2018; Organoids: Stem Cells, Structure, and Function (Methods in Molecular Biology, 1576), Springer, 1st edition, 2019; and Human Pluripotent Stem Cell Derived Organoid Models (Volume 159) (Methods in Cell Biology, Volume 159), Academic Press, 1^{st} edition, 2020; herein incorporated by reference in their entireties.

In certain embodiments, a non-human organism is grown in a cell culture well. Non-human organisms may include, for example, without limitation, bacteria, archaea, protists, fungi, and algae.

The growth of cultures may be confirmed by any convenient method, e.g., phase contrast microscopy, stereomicroscopy, histology, immunohistochemistry, electron microscopy, fluorescence microscopy, etc. In some instances, cellular ultrastructure and multi-lineage differentiation may be assessed. Ultrastructure of a culture can be determined by performing hematoxylin-eosin staining, proliferating cell nuclear antigen (PCNA) staining, electron microscopy, and the like using methods known in the art. Multi-lineage differentiation can be determined by performing labeling with antibodies to terminal differentiation markers. Antibodies to detect differentiation markers are commercially available from a number of sources.

In some embodiments, the cells in cultures may be experimentally modified. For example, cells may be modified by exposure to viral or bacterial pathogens, e.g., to develop a reagent for experiments to assess the anti-viral or anti-bacterial effects of therapeutic agents. The cells may be modified by altering patterns of gene expression, e.g., by providing reprogramming factors to induce pluripotency or otherwise alter differentiation potential, or to determine the effects of a gain or loss of gene function.

Experimental modifications may be made by any method known in the art, for example, as described below with regard to methods for providing candidate agents that are nucleic acids, polypeptides, small molecules, viruses, etc. to cells for screening purposes.

### Screening

A culture, for example, of a cell, a population of cells, a tissue, an organoid, or a non-human organism, grown in the chamber of a cell culture well, may be analyzed to determine its response to exposure to a test agent. Candidate test agents may include, without limitation, pharmaceutical agents such as small molecules, drugs, chemotherapeutic agents, biologic agents, and immunotherapeutic agents, antibodies, peptides, proteins, secreted factors such as growth factors, cytokines, chemokines, and hormones, and genetic agents such as antisense nucleic acids, miRNA, siRNA, shRNA, mRNA, cDNA, CRISPR systems, vectors encoding expressible sequences, toxins, pathogenic agents such as viruses, bacteria, fungi, protists, and the like. In some embodiments, a cell, population of cells, tissue, organoid, or non-human organism grown in a cell culture well, as described herein, is used as a disease model to determine the effects of a candidate agent for treating a disease.

The culture may be contacted with agents by any convenient means. Generally, the agents are added to the culture media used for growth of the cultures such that the agent is brought in contact with the cells at an effective concentration to produce a desired effect. For example, the agents can be added to a culture by flowing media containing the agent through the inlet channel into a chamber. In some embodiments, a plurality of candidate agents for screening are stored in one or more reservoirs connected to one or more inlet channels to allow screening of a plurality of cultures in different chambers in parallel with the same or different candidate agents. Alternatively, agents can be injected into the culture, e.g., through the top opening into the chamber, and their effects compared to injection of controls.

The effective concentration of an agent will vary and will depend on the agent. In some instances, the effective concentration may also depend on the type of culture (e.g., cells, tissue, organoid, or organism), the culture conditions, or other agents present in the culture media, etc. In some embodiments, the effective concentration of agents ranges from 1 ng/ml to 10 mg/mL or more, including but not limited to, e.g., 1 ng/mL, 2 ng/ml, 3 ng/mL, 4 ng/ml, 5 ng/ml, 6 ng/mL, 7 ng/ml, 8 ng/mL, 9 ng/ml, 10 ng/mL, 11 ng/mL, 12 ng/mL, 13 ng/ml, 14 ng/mL, 15 ng/mL, 16 ng/mL, 17 ng/mL, 18 ng/mL, 19 ng/ml, 20 ng/mL, 21 ng/ml, 22 ng/ml, 23 ng/ml, 24 ng/mL, 25 ng/mL, 26 ng/mL, 27 ng/mL, 28 ng/ml, 29 ng/ml, 30 ng/ml, 31 ng/mL, 32 ng/mL, 33 ng/ml, 34 ng/ml, 35 ng/mL, 36 ng/ml, 37 ng/mL, 38 ng/ml, 39 ng/ml, 40 ng/mL, 41 ng/mL, 42 ng/mL, 43 ng/mL, 44 ng/mL, 45 ng/mL, 46 ng/ml, 47 ng/ml, 48 ng/mL, 49 ng/mL, 50 ng/mL, 1-5 ng/mL, 1-10 ng/mL, 1-20 ng/mL, 1-30 ng/mL, 1-40 ng/mL, 1-50 ng/mL, 5-10 ng/mL, 5-20 ng/mL, 10-20 ng/mL, 10-30 ng/mL, 10-40 ng/ml, 10-50 ng/ml, 20-30 ng/mL, 20-40 ng/mL, 20-50 ng/ml, 30-40 ng/ml, 30-50 ng/mL, 40-50 ng/mL, 1-100 ng/mL, 50-100 ng/mL, 60-100 ng/mL, 70-100 ng/mL, 80-100 ng/mL, 90-100 ng/ml, 10-100 ng/mL, 50-200 ng/mL, 100-200 ng/mL, 50-300 ng/mL, 100-300 ng/mL, 200-300 ng/mL, 50-400 ng/mL, 100-400 ng/mL, 200-400 ng/mL, 300-400 ng/mL, 50-500 ng/mL, 100-500 ng/mL, 200-500 ng/mL, 300-500 ng/mL, 400 to 500 ng/mL, 0.001-1 μg/mL, 0.001-2 μg/mL, 0.001-3 μg/mL, 0.001-4 μg/mL, 0.001-5 μg/mL, 0.001-6 μg/mL, 0.001-7 μg/mL, 0.001-8 μg/mL, 0.001-9 μg/mL, 0.001-10 μg/mL, 0.01-1 μg/mL, 0.01-2 μg/mL, 0.01-3 μg/mL, 0.01-4 μg/mL, 0.01-5 μg/mL, 0.01-6 μg/mL, 0.01-7 μg/mL, 0.01-8 μg/mL, 0.01-9 μg/mL, 0.01-10 μg/mL, 0.1-1 μg/mL, 0.1-2 μg/mL, 0.1-3 μg/mL, 0.1-4 μg/mL, 0.1-5 μg/mL, 0.1-6 μg/mL, 0.1-7 μg/mL, 0.1-8 μg/mL, 0.1-9 g/mL, 0.1-10 μg/mL, 0.5-1 μg/mL, 0.5-2 μg/mL, 0.5-3 μg/mL, 0.5-4 μg/mL, 0.5-5 μg/mL, 0.5-6 μg/mL, 0.5-7 μg/mL, 0.5-8 μg/mL, 0.5-9 μg/mL, 0.5-10 μg/mL, 0.1 mg/mL-10 mg/mL, 0.1 mg/mL-1 mg/mL, 1 mg/mL-9 mg/mL, 2 mg/mL-8 mg/mL, 3 mg/mL-7 mg/mL, and the like.

The effect of an agent on cultures is determined by adding the agent to the culture and monitoring one or more parameters usually with comparison to a control culture lacking the agent. The parameters may include, without limitation, growth, differentiation, gene expression, proteome, phenotype with respect to markers etc. of the cells, e.g., any cell component or cell product including cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. While most parameters will provide a quantitative readout, in some instances a semi-quantitative or qualitative result will be acceptable. Readouts may include a single determined value, or may include a mean, median value or variance, etc. Characteristically a range of parameter readout values will be obtained for each parameter from a multiplicity of the same assays. Some variability is expected and a range of values for each set of test parameters may be obtained and analyzed using standard statistical methods.

In some embodiments, candidate agent is added to the cells within an intact tissue or organoid. In other embodiments, tissues or organoids are dissociated, and the candidate agent is added to the dissociated cells. The cells may be freshly isolated, cultured, or genetically altered as described above. The cells may be environmentally induced variants of clonal cultures: e.g., split into independent cultures and grown into tissues or organoids under distinct conditions. The manner in which cells respond to an agent, particularly a pharmacologic agent, including the timing of responses may reflect the physiologic state of the cells.

Candidate agents of interest for screening include known and unknown compounds that encompass numerous chemical classes, primarily organic molecules, which may include organometallic molecules, inorganic molecules, genetic sequences, etc.

Candidate agents may include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, proteins, antibodies, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, immunotherapeutic agents, biologic agents, neuropeptides, hormones, agonists, or antagonists, etc. Exemplary pharmaceutical agents include those described in, “The Pharmacological Basis of Therapeutics,” Goodman and Gilman, McGraw-Hill, New York, N. Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g., as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the cultures are contacted with vectors comprising the nucleic acid of interest such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. For example, the cells are contacted with viral particles comprising the nucleic acid of interest. Retroviruses, for example, lentiviruses, are particularly suitable. Commonly used retroviral vectors are “defective”, i.e., unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90: 8392-8396). Retroviruses bearing amphotropic envelope protein, e.g., 4070A are capable of infecting most mammalian cell types, including human, dog and mouse, and are generated by using amphotropic packaging cell lines such as PA12 (Miller et al. (1985) Mol. Cell. Biol. 5: 431-437); PA317 (Miller et al. (1986) Mol. Cell. Biol. 6: 2895-2902); GRIP (Danos et al. (1988) PNAS 85: 6460-6464). Retroviruses packaged with xenotropic envelope protein, e.g., AKR env, are capable of infecting most mammalian cell types, except murine cells. The appropriate packaging cell line may be used to ensure that the cultured cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing a nucleic acid of interest to the subject cells will typically comprise suitable promoters for driving the expression, that is, transcriptional activation, of the nucleic acid of interest. This may include ubiquitously acting promoters, for example, the CMV-B-actin promoter, or inducible promoters, such as promoters that are active in particular cell populations or that respond to the presence of drugs such as tetracycline. By transcriptional activation, it is intended that transcription will be increased above basal levels in the target cell by at least about 10-fold, by at least about 100-fold, more usually by at least about 1000-fold. In addition, vectors may include genes that are later be removed, e.g., using a recombinase system such as Cre/Lox. Vectors may include genes that confer selective toxicity such as herpesvirus TK, bcl-xs, etc., to allow destruction of the cells

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one culture or cell sample, usually in conjunction with a control culture or cell sample that is not contacted with the agent. Changes in parameters in response to the agent are measured, and the result is evaluated by comparison to reference cultures, which may include cultures in the presence or absence of the agent, or treated with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow-through method. Alternatively, the agents can be injected into the culture, e.g., into the chamber, and their effects compared to injection of controls.

Preferred agent formulations do not include additional components, such as preservatives, that may have a significant effect on the overall formulation. Thus, preferred formulations consist essentially of a biologically active compound and a physiologically acceptable carrier, e.g., water, ethanol, DMSO, etc. If a compound is a liquid, it may not require a solvent, and the formulation may consist essentially of the compound itself.

A plurality of assays may be run in parallel with different agent concentrations to obtain a differential response to the various concentrations. As known in the art, determining the effective concentration of an agent typically involves testing a range of concentrations resulting from 1:10, or other log scale, dilutions. The amount of an agent needed to be effective may be further refined with a further series of dilutions, if necessary. A control may include the agent at zero concentration, at a concentration below the level of detection of the agent, or at a concentration that does not give a detectable change in the growth rate or other parameter used to monitor a culture.

A candidate agent may also be screened for efficacy in treating or preventing a disease. In such embodiments, the culture models the disease. For example, cells or tissue may have been obtained from a diseased tissue or may be experimentally modified to model a disease by, e.g., genetic mutation. Screening may involve monitoring parameters such as cell growth, cell viability, cell ultrastructure, tissue ultrastructure, and the like.

A candidate agent may also be screened for toxicity to cells or tissue. In these applications, a culture is exposed to a candidate agent and its growth and viability are assessed.

In some embodiments, methods and culture systems are provided for screening candidate agents in a high-throughput format. By “high-throughput” is meant the screening of large numbers of candidate agents simultaneously for an activity of interest. By large numbers, it is meant screening 20 more or candidates at a time, e.g., 40 or more candidates, e.g., 100 or more candidates, 200 or more candidates, 500 or more candidates, or 1000 candidates or more.

In some embodiments, the high-throughput screen will be formatted based upon the numbers of wells in the cell culture plates used, e.g. a 24-well format, in which 24 candidate agents (or less, plus controls) are assayed; a 48-well format, in which 48 candidate agents (or less, plus controls) are assayed; a 96-well format, in which 96 candidate agents (or less, plus controls) are assayed; a 384-well format, in which 384 candidate agents (or less, plus controls) are assayed; a 1536-well format, in which 1536 candidate agents (or less, plus controls) are assayed; or a 3456-well format, in which 3456 candidate agents (or less, plus controls) are assayed.

### Kits

Also provided are kits comprising a cell culture well, a cell culture plate, or a microfluidic device, as described herein. In certain embodiments, the cell culture plate or microfluidic device comprises at least 2, at least 4, at least 6, at least 12, at least 24, at least 48, at least 96, at least 384, or at least 1536 cell culture wells. In some embodiments, the cell culture well, cell culture plate, or microfluidic device is contained in a sterile package. In some embodiments, the kit includes multiple cell culture wells, cell culture plates, or microfluidic devices. The kit may also include culture media, incubators, microscopes, and/or other reagents or equipment for culturing, for example, a cell, a population of cells, a tissue, an organoid, or a non-human organism.

In addition to the above components, the subject kits may further include (in certain embodiments) instructions for practicing the subject methods. In some embodiments, instructions for using the cell culture well, cell culture plate, or microfluidic device are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, SD drive, and the like, on which the information has been recorded. Yet another form of these instructions that may be present is a website address which may be used via the internet to access the information at a removed site.

### Examples of Non-Limiting Embodiments of the Disclosure

Notwithstanding the appended claims, the present disclosure is also defined by the following embodiments.
1. A cell culture well, comprising:
   a chamber comprising a bottom surface and a top opening;
   an inlet channel in fluid communication with the chamber, wherein the inlet channel connects with the chamber at the top opening at a first side of the chamber, wherein a first segment of the inlet channel is substantially perpendicular to the first side at the bottom surface and wherein a second segment of the inlet channel is substantially parallel to the first side of the chamber from the top opening to the bottom surface; and
   an outlet channel in fluid communication with the chamber, wherein the outlet channel connects with the chamber at the top opening, wherein a first segment of the outlet channel is substantially parallel to a second side of the chamber from the top opening to the bottom surface, and wherein a second segment of the outlet channel is substantially perpendicular to the second side at the bottom surface.
2. The cell culture well of embodiment 1, wherein the inlet channel is configured to be substantially perpendicular to the first side for a first distance and substantially parallel to the first side at the end of the first distance and wherein the outlet channel is configured to be substantially perpendicular to the second side for a second distance and substantially parallel to the first side at the end of the first distance.
3. The cell culture well of embodiment 1 or 2, wherein the first side comprises a first ramp portion on top of the first side, and the second side comprises a second ramp portion on top of the second side, wherein the first ramp portion forms an obtuse angle relative to the first side and the second ramp portion forms an obtuse angle relative to the second side.
4. The cell culture well of any one of embodiments 1-3, further comprising a cap, wherein the top opening is covered with the cap.
5. The cell culture well of embodiment 4, wherein the cap is removable.
6. The cell culture well of any one of embodiments 1-5, wherein the bottom surface is transparent.
7. A cell culture plate comprising at least two of the cell culture wells of any one of embodiments 1-6.
8. The cell culture plate of embodiment 7, wherein the inlet channel of a first cell culture well is in fluid communication with the inlet channel of a second cell culture well.
9. The cell culture plate of embodiment 7 or 8, wherein the outlet channel of the first cell culture well is in fluid communication with the outlet channel of the second cell culture well.
10. The cell culture plate of embodiments 7-9, wherein the cell culture plate comprises at least 2, at least 4, at least 6, at least 12, at least 24, at least 48, at least 96, at least 384, or at least 1536 cell culture wells.
11. A microfluidic device comprising the cell culture plate of any one of embodiments 7-10.
12. The microfluidic device of embodiment 11, further comprising a container for storage of culture media.
13. The microfluidic device of embodiment 11 or 12, further comprising a container for collection of waste.
14. The microfluidic device of any one of embodiments 11-13, further comprising a pump.
15. The microfluidic device of any one of embodiments 11-14, further comprising a multiport fluid distribution valve.
16. The microfluidic device of any one of embodiments 11-15, further comprising a flow rate sensor.
17. The microfluidic device of any one of embodiments 11-16, further comprising a temperature sensor.
18. A method of using the cell culture well of any one of embodiments 1-6, the method comprising:
   introducing a cell, a population of cells, a tissue, an organoid, or a non-human organism into the chamber through the top opening;
   introducing a first culture media into the inlet channel;
   flowing the first culture media through the inlet channel into the chamber; and
   culturing the cell, population of cells, tissue, organoid, or non-human organism in the first culture media under conditions suitable for growth of the cell, tissue, organoid, or non-human organism.
19. The method of embodiment 18, further comprising removing the first culture media from the chamber by flowing the culture media through the outlet channel.
20. The method of embodiment 19, further comprising flowing the first culture media out of the outlet channel into a waste container.
21. The method of embodiment 20, further comprising adding a second culture media to the chamber by flowing the second culture media through the inlet channel into the chamber.
22. The method of any one of embodiments 18-21, further comprising adding a media supplement to the chamber by flowing the media supplement through the inlet channel into the chamber.
23. The method of any one of embodiments 18-22, further comprising removing the cell, population of cells, tissue, organoid, or non-human organism from the chamber.
24. The method of any one of embodiments 18-23, further comprising imaging the cell, population of cells, tissue, organoid, or non-human organism.
25. The method of any one of embodiments 18-24, further comprising performing expression profiling or a biochemical assay on the cell, tissue, organoid, or non-human organism.
26. A method of making the cell culture well of embodiment 1, the method comprising:
   providing a first layer comprising a set of channels parallel to the plane of the first layer; and
   adding on top of the first layer, a second layer, the second layer comprising a hole, an inlet chamber in fluid communication with an end of the hole configured to be furthest from the first layer and an outlet channel in fluid communication with the end of the hole configured to be furthest from the first layer.
27. An injection molding for fabricating a cell culture well, the injection molding comprising a substrate surrounding a cavity having a configuration as illustrated in FIG. 5, wherein material injected into the cavity hardens to assume the configuration of the cavity to form the molded cell culture well of embodiment 1.

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1: Microfluidic Well Plate for High Throughput 3D Cell Culture

### 1. Automated Cell Culture Overview

Systems that perform automated cell culture can enhance pharmacology, pathology, and basic research by improving the quality and consistency of cultures while reducing reagent consumption and required labor. Platforms particularly useful for medical studies are those that support 3D culturing of cells (organoids) or patient tissue samples. Described here is the development of such an automated culture system with a plurality of channels and wells for high throughput 3D cell culture. This system comprises media storage, fluid pumps, fluid valve manifolds, a microfluidic chip containing numerous wells/bioreactors with individual media and waste lines, and a conditioned media capture system (FIG. 2).

### 2. Context

Liquid dispensing type systems typically fall into two broad categories, i.e., gantry/robotic feeding systems (robotic pipetting) versus microfluidic chip systems (the disclosed system) and have differences that depend on the type of biology the system is designed for (e.g., organ-on-chip, body-on-chip, or organoid/spheroid). These categories impact the overall design of the cell culture chip and system. For example, a system with manual or robotic pipetting will typically require that the well have an open top or easily removable lid for the pipette to access, while a microfluidic system often includes serially connected wells to reduce the number of fluid valves required to address each well individually or on-chip valves or pumps to enable microfabricated valve techniques that scale. A robotic pipette can perform automated culture on traditional 24, 96, 384, 1536-well plates wherein each well is typically no more than a simple cylindrical vessel while a microfluidic system requires custom multi-layer well plates with integrated microfluidics and valves that can be very costly. Ultimately, while the microfluidic approach for solving culture automation from a biological perspective is preferable as it more closely recreates the biological environment, the cost to manufacture a chip can be limiting for many teams. Therefore, minimizing the cost to produce a microfluidic well plate by minimizing the number of manufacturing steps is desirable.

The specific biology for which a system was designed can also significantly impact the chip design. Organ-on-chip technology typically involves multiple layers of different cell types that are cultured on different surfaces/scaffolds such as porous membranes or hydrogels that mimic the apical and basal surfaces of organs. Organoid/spheroid technology involves the culture self-differentiating clusters of cells that can turn into different tissue types; use of a well or bioreactor to produce these structures can be simpler than organ-on-chip technology. Although the use of porous membranes as scaffolds is limited in organoid/spheroid cultures, hydrogel coatings can be used to support the free-standing 3D culture. Body-on-chip technology involves organ models mimicking different tissues and can further involve serially connected organoid wells/bioreactors, organ-on-chip models, or both in fluid communication with one another.

### 3. Organoid Well Plates

The disclosed design can be used with organoid/spheroid, organ-on-chip, and body-on-chip systems, or any other culture system including explant tissues, 2D cultures and non-human organisms (e.g. aquatic organisms, yeast, and plants).

Intended outcomes for the disclosed systems include:
1. To produce a system for exchanging media that minimizes fluid velocity and/or turbulence for the biology present as to not disturb delicate microstructure or move the samples and thereby produce spatial inconsistencies in serial imaging.
2. To produce a system that is parallelizable, enabling high throughput assays.
3. To produce the well plates in the system at as low a cost as possible by minimizing the number of steps involved in manufacturing.
4. To produce wells that have a transparent bottom enabling imaging from below the plate.

To achieve these outcomes, provided herein is a microfluidic plate with a 3D geometry that can support cell culture in one or more different wells (FIG. 3A). By putting the inlets at the top of the well, the inlets are spaced as far as possible from the biological material. This slows the flow over the biological material and in turn minimizes forces acting on the biological material that can stress delicate structures and systems. In more specific applications, the angle and profile of the inlet and outlet geometries may be designed to further manipulate or eliminate any vortices that form when fluid is flowing, such as when fresh media displaces old media, thereby creating concentration gradients over the sample.

The plate can be manufactured in a two-step process: (a) manufacturing a machined layer defining the microchannels, and wells, and (b) bonding the machined layer to a substrate to seal the microchannels (FIG. 3B). This process is simplified by the vertical channels that connect the fluidics at the bottom of the plate to the inlet at the top of the well. When the channel connects to the surface of the plate, the inlet to the well is sloped from the top of the plate to the well itself (FIGS. 3C, 3D). As a result, the entire fluidic component can be formed in a single process (e.g. injection molding or 3-D printing). The simplicity of manufacture stands in stark contrast to microfluidics systems with fully enclosed 3D microfluidics.

The above examples are provided to illustrate the invention but not to limit its scope. Other variants of the invention will be readily apparent to one of ordinary skill in the art and are encompassed by the appended claims. All publications, accessions, references, databases, and patents cited herein are hereby incorporated by reference for all purposes.

What is claimed is:

1. A cell culture well, comprising:
a chamber comprising a bottom surface and a top opening;
an inlet channel in fluid communication with the chamber, wherein the inlet channel connects with the chamber at the top opening at a first side of the chamber, wherein a first segment of the inlet channel is substantially perpendicular to the first side at the bottom surface and wherein a second segment of the inlet channel is substantially parallel to the first side of the chamber from the top opening to the bottom surface; and
an outlet channel in fluid communication with the chamber, wherein the outlet channel connects with the chamber at the top opening, wherein a first segment of the outlet channel is substantially parallel to a second side of the chamber from the top opening to the bottom surface, and wherein a second segment of the outlet channel is substantially perpendicular to the second side at the bottom surface.

2. The cell culture well of claim 1, wherein the inlet channel is configured to be substantially perpendicular to the first side for a first distance and substantially parallel to the first side at the end of the first distance and wherein the outlet channel is configured to be substantially perpendicular to the second side for a second distance and substantially parallel to the first side at the end of the first distance.

3. The cell culture well of claim 1, wherein the first side comprises a first ramp portion on top of the first side, and the second side comprises a second ramp portion on top of the second side, wherein the first ramp portion forms an obtuse angle relative to the first side and the second ramp portion forms an obtuse angle relative to the second side.

4. The cell culture well of claim 1, further comprising a cap, wherein the top opening is covered with the cap.

5. The cell culture well of claim 4, wherein the cap is removable.

6. The cell culture well of claim 1, wherein the bottom surface is transparent.

7. A cell culture plate comprising at least two of the cell culture wells of claim 1.

8. The cell culture plate of claim 7, wherein the inlet channel of a first cell culture well is in fluid communication with the inlet channel of a second cell culture well.

9. The cell culture plate of claim 7, wherein the outlet channel of the first cell culture well is in fluid communication with the outlet channel of the second cell culture well.

10. The cell culture plate of claim 7, wherein the cell culture plate comprises at least 4 of the cell culture wells.

11. A microfluidic device comprising the cell culture plate of claim 7.

12. The microfluidic device of claim 11, further comprising one or any combination of:
a container for storage of culture media;
a container for collection of waste;
a pump;
a multiport fluid distribution valve;
a flow rate sensor; and
a temperature sensor.

13. A method of using the cell culture well of claim 1, the method comprising:
introducing a cell, a population of cells, a tissue, an organoid, or a non-human organism into the chamber through the top opening;
introducing a first culture media into the inlet channel;
flowing the first culture media through the inlet channel into the chamber; and
culturing the cell, population of cells, tissue, organoid, or non-human organism in the first culture media under conditions suitable for growth and/or differentiation of the cell, tissue, organoid, or non-human organism.

14. The method of claim 13, further comprising removing the first culture media from the chamber by flowing the culture media through the outlet channel.

15. The method of claim 14, further comprising adding a second culture media to the chamber by flowing the second culture media through the inlet channel into the chamber.

16. The method of claim 13, further comprising adding a media supplement to the chamber by flowing the media supplement through the inlet channel into the chamber.

17. The method of claim 13, further comprising removing the cell, population of cells, tissue, organoid, or non-human organism from the chamber.

18. The method of claim 13, further comprising imaging and/or performing expression profiling or a biochemical assay on the cell, population of cells, tissue, organoid, or non-human organism.

19. A method of making the cell culture well of claim 1, the method comprising:
providing a first layer comprising a set of channels parallel to the plane of the first layer; and
adding on top of the first layer, a second layer, the second layer comprising a hole, an inlet chamber in fluid communication with an end of the hole configured to be furthest from the first layer and an outlet channel in fluid communication with the end of the hole configured to be furthest from the first layer.

20. An injection molding for fabricating a cell culture well, the injection molding comprising a substrate surrounding a cavity having a configuration as illustrated in FIG. 5, wherein material injected into the cavity hardens to assume the configuration of the cavity to form the molded cell culture well of claim 1.
